(19) United States
(12) Patent Application Publication
(10) Pub. No.: US 2010/0203088 A1
(43) Pub. Date: Aug. 12, 2010

(54) Silver Nanoparticle Dispersion Formulation

(76) Inventors: Khandelwal Sanjeev, Mumbai (IN); Omray Pratibha, Mumbai (IN)

Correspondence Address: Charles Muserlain, 317 Bliss Lane, Valley Cottage, NY, 10989, US

(21) Appl. No.: 11/921,970

(22) PCT Filed: Jun. 8, 2006
(86) PCT No.: PCT/IN2006/000193

§ 371 Date: Feb. 13, 2009

(30) Foreign Application Priority Data
Jun. 8, 2005 (IN) 688/MUM/05

(51) Int. Cl. A61K 9/00; A61K 33/38; A61P 17/02

(52) U.S. Cl. 424/400; 424/618; 977/773

(57) ABSTRACT

A topical semisolid (gel) formulation of nanoparticles of silver comprising about 0.001% w/w to about 1% w/w of silver by weight of the total mass of the formulation with its particle size in the range of 0.1 to 150 nanometer; about 0.5% w/w to about 25% w/w of at least one gel forming agent by weight of the total mass of the formulation; about 25% w/w to 60% w/w of at least of one co-solvent by weight of the total mass of the formulation; about 1% w/w to 50% w/w of purified water by weight of the total mass of the formulation and other optional adjuvants wherein the nanoparticle size of the silver enhances the therapeutic action of the formulation.

### FIELD OF INVENTION

This invention relates to a semisolid formulation containing silver. The invention envisages silver dispersed in the form of fine particles in the range of nanometre size particles and meant to be used for topical application for various infections. This invention further envisages the production of formulations comprising silver as nanoparticles in dispersion.

### INTRODUCTION

The use of silver in human health care & medicine is very old and it was used in ancient time as Disinfectant, Antiseptic, Dentistry, Wound therapy

Presently, silver is used in the form of a salt or in the form of colloidal solution. Recently various medical grade advanced technologies have been developed for safer and bioavailable compounds.

In Ayurveda, it is reported that silver cure skin problems, weight loss, Anemia, night blindness, burns and is bactericidal.

In allopathy it is already reported to prevent and control the spread of infections for wound and burn healing.

In developing topical antimicrobial pharmaceutical compositions consisting of two different antimicrobial agents, ionized silver has been a preferred agent. Silver, in its ionic state, is inherently safe and possesses a very broad spectrum of antimicrobial efficacy. Specifically, ionized silver has broad antibacterial, antifungal and antiviral properties. N. Grier, “Silver and Its Compounds” in Disinfection, Sterilization, and Preservation, (3rd edition S. S. Block, ed.), Lea & Febiger, Philadelphia, Ch. 20, p. 395 (1983). Additionally, being oligodynamic, ionized silver can provide long-lasting, or “residual”, antimicrobial protection.

The broad spectrum of antimicrobial activity of ionized silver is caused by the reactivity of silver ions with a variety of functional groups. Silver ions, similar to most heavy metals in their ionized state, can complex with electron-donating functional groups containing sulfur, oxygen or nitrogen. In biological systems these electron donor groups are present as functional groups such as thiols, carboxylates, phosphates, hydroxyl, amines, imidazoles and indoles, either singly or in many varied combinations. These electron donor groups are found in great numbers in a variety of biomolecules, which make up microbes. The binding of ionized silver to any of these electron donor groups causes disruption or inactivation of the biological system, resulting in the microbe's death. Depending on the source of the silver ions, studies indicate that silver ions kill the microbe either by attacking the cell wall and membrane producing blebs or by producing aggregation of nuclear material into filaments. N. Grier, “Silver and Its Compounds” in Disinfection, Sterilization, and Preservation, (3rd edition. S. S. Block, ed.), Lea & Febiger, Philadelphia, Ch. 20, p. 395 (1983).

The medical use of ionized silver has been limited to the use of silver nitrate because of silver nitrate's ability to completely ionize in water. Silver nitrate solutions (1%) have been used as eye drops in newborn babies for years to prevent ophthalmia neonatorum. In addition, dressings wetted with 0.5% silver nitrate solution have been used to cover second- and third-degree burns to prevent and treat infections. Unfortunately, silver nitrate solutions are very photo-unstable and leave a dark stain on anything with which they come into contact; therefore, they are not widely utilized.

### BACKGROUND OF THE INVENTION

U.S. Pat. No. 3,092,552 discloses the use of silver ions as an oligodynamic agent in a therapeutic or surface-treating composition or as a means for germicidaily protecting an article or surface. Specifically, the disclosed composition comprises a low concentration of a silver compound such as silver nitrate or silver oxide, a reducing agent such as starch or sugar, polyethylene glycol (PEG), and urea. Though the patent teaches that the addition of small amounts of sodium chloride or cupric chloride to the composition prevents discoloration, even when the product is exposed to sterilization procedures and direct sunlight, it has been demonstrated that the silver ion compositions are not photostable.

U.S. Pat. No. 3,761,590 attempted to improve on the shortcomings of silver nitrate solutions by complexing the silver ion to the antibiotic sulfadiazine to form silver sulfadiazine. Silver sulfadiazine is a non-ionized, water-insoluble powder which is administered as a 1% cream to prevent bacterial infection in the treatment of burns. >N. Grier, “Silver and Its Compounds” in Disinfection, Sterilization, and Preservation, (3rd edition S. S. Block, ed.), Lea & Febiger, Philadelphia, Ch. 20, p. 395 (1983). While compositions containing silver sulfadiazine are nonstaining and have greatly improved photostability over ionized silver, they are less than ideal because the silver is in the form of a silver salt and not in the form of ionized silver. As a result, silver ions must be ionized off the silver sulfadiazine salt powder in order to be antimicrobially active.

From the above, it should be clear that the presently available silver-based antimicrobial compositions such as silver salts have the side effects. In the mean while it is reported that the silver has an excellent therapeutic antimicrobial activity. Thus there is a need for a suitable formulation, which can alternatively enhance the reported action of the silver avoiding the side effects those of silver salts.

### OBJECT OF THE INVENTION

The object of the invention is to provide a semisolid formulation of silver wherein the silver is dispersed in the form of fine particles in the range of nanometre size particles and meant to be used for topical application for various infections.

Yet another object of the invention is to provide a process for the preparation of formulation comprising silver nanoparticles dispersion.

### SUMMARY OF THE INVENTION

According to this invention, therefore there is provided a topical semisolid (gel) formulation comprising,
a) therapeutically effective amount of silver nanoparticles in the range of about 0.1 nanometer to about 150 nanometer having mass of about 0.001% w/w to about 1% w/w of the formulation;
b) at least one gel forming agent (viscosity enhancing polymer) having mass of about 0.5% w/w to about 25% w/w of the formulation;
c) at least one co-solvent (penetrating enhancing agents) having mass of about 25% w/w to 60% w/w of the formulation;
d) purified water having mass of about 1% w/w to 50% w/w of the formulation;
e) optionally at least one preservative having mass of about 0.001% w/w to 2.0% w/w of the formulation;
f) optionally at least one antioxidant having mass of about 0.001% w/w to 2.0% w/w of the formulation;
g) optionally at least one pH adjusting agent having mass of about 0.01% w/w to 1.0% w/w of the formulation;
h) optionally at least one fragrance having mass of about 0.01% w/w to 1.0% w/w of the formulation.

In accordance with one embodiment of the invention, the silver nanoparticles are in the form of suspension.

In accordance with another embodiment of the invention, semisolid formulation is used for local administration to the infected area of the skin or the oral cavity on the microbial infections in cuts, wound, burns and other topical microbial infection.

In accordance with another embodiment of the invention, the gel forming agent is a compound selected from a group of compounds consisting of carbomers [cross linked polymers of acrylic acid], cellulosic polymers such as hydroxypropyl methylcellulose, methylcellulose, sodium carboxy methylcellulose and hydroxypropyl cellulose.

In accordance with another embodiment of the Invention, the cosolvent is at least one compound selected from a group of compounds consisting of Propylene glycol, polyethylene glycols, cremaphors, ethyl alcohol, isopropyl alcohol and Polysorbate.

In accordance with another embodiment of the invention, the preservative is at least one compound selected from a group of compounds consisting of chlorocresol, methyl paraben, propyl paraben, thiomarsal, sorbic acid, potassium sorbate and benzyl alcohol.

In accordance with another embodiment of the invention, the antioxidant is at least one compound selected from a group of compounds consisting of Butylated hydroxy anisole (BHA), Butylated hydroxy toluene (BHT), sodium metabisulfite, sodium sulfite, sodium bisulfite and propyl gallate.

In accordance with another embodiment of the invention, the pH adjusting agent is at least one compound selected from a group of compounds consisting of diethanolamine, triethanolamine, sodium hydroxide, hydrochloric acid, citric acid and mono basic sodium phosphate.

In accordance with another embodiment of the Invention, the pH of the formulation is in the range of 4.5 to 6.5.

In accordance with another aspect of the invention, there is provided a process for preparing a topical semisolid (gel) formulation comprising the following steps
a) Dispersing a gel forming agent in a mixture of cosolvent and purified water in a container and mixing vigorously at temperatures in the range of 25 to 70° C. and after complete dispersion cooling the solution to get a jelly mass,
b) Adding silver suspension to the jelly mass with mixing till the silver suspension is uniformly distributed through out the content of the mixture to get a silver containing jelly,
c) Dissolving preservative, antioxidant, fragrance in the purified water and/or isopropyl alcohol to get an excipients solution,
d) adding the excipients solution to the silver containing jelly with stirring and adjusting the pH to between 4.5 to 6.5 and the volume were with the solution of pH adjusting agents and purified water respectively.

The present invention comprises a semisolid formulation containing silver wherein the silver is in the form of silver suspension having particle size in the nanometer range. This very small particle size of the silver helps to improve the antimicrobial activity. The silver present in the semisolid formulation in a fine dispersion form and stable throughout the shelf life of the formulation and has no side effect as that of silver salts. The comparative in-vitro data of the present formulation with that of silver salt formulation supports contentions that present formulation has excellent antimicrobial activity and with minimum inhibitory concentration (MIC) than the silver salt.

### DETAILED DESCRIPTION OF THE INVENTION

The main feature of this invention is the making the topical semisolid dosage form (gel) of the formulation comprising silver as a nano particle dispersion and a method of making the formulation. The particle size of silver present in the formulation varies from 0.1 nanometer to 150 nanometers. The silver nanoparticles present in the formulation are dispersed in the aqueous medium and may be termed as silver nano suspension. This silver nano suspension can be used for the preparation of the nano topical gel for external application or for infection in the mouth cavity in any pharmaceutical form may be as film, patches, tablet, gel or can be administered to the gastrointestinal tract.

The semisolid gel formulations have significant and improved effect than the silver salt formulations.

The composition of semisolid pharmaceutical formulation comprising
(a) 0.001 w/w to 1% w/w of silver.
(b) 0.5% w/w to 25% w/w of gel forming agent (polymers)
(c) 25% w/w to 60% w/w of co-solvents or penetrating enhancing agents.
(d) 0.001 w/w to 2.0% w/w of preservatives and/or antioxidants (optional).
(e) 0.01% w/w to 1.0% w/w of fragrance (optional)
(f) other pharmaceutical acceptable inert excipients such as solvents and pH adjusting agents,
(g) other pharmaceutical additives optionally added such as colors, taste modifiers etc,

The said formulation may contain one or more pharmaceutical acceptable inert excipients comprising of preservatives, antioxidants, surfactants and pH adjusting agents.

The preservative in the formulation is optionally used. It may comprise chlorocresol, methyl paraben, propyl paraben, thiomarsal, sorbic acid, potassium sorbate and benzyl alcohol. The preferable concentration of these preservatives is 0.001% w/w to 2% w/w.

The antioxidants are optionally used in the formulation may includes Butylated hydroxy anisole (BHA), Butylated hydroxy toluene (BHT), sodium metabisulfite, sodium sulfite, sodium bisulfite and propyl gallate.

The surfactants used in the formulation mainly to improve the penetration of the active ingredients to the skin by increasing the solublization of the active in the lipophilic membrane. These may include Polysorbate 80, Polysorbate 20, sorbitan groups (spans). The concentration of surfactant in the formulation may include 0.1% w/w to 3.0% w/w.

The fragrance in the formulation may be any pharmaceutically acceptable perfumes and used mainly to add an appeal to the product.

The pH adjusting agents used in the formulation may include diethanolamine, triethanolamine, sodium hydroxide, hydrochloric acid, citric acid and mono basic sodium phosphate.

Semisolid base in the formulation mainly comprising of the adjuvant solvents, viscosity enhancer (jelling) agents and solvent such as isopropyl alcohol and purified water.

The cosolvent in the formulation includes glycols that may be selected from the Propylene glycol, polyethylene glycols [PEG-300, PEG-400, etc], and other substituted glycols such as different grade of cremaphors and other cosolvent in the formulation may include the ethyl alcohol, isopropyl alcohol, Polysorbate and other surfactants.

The viscosity increasing agents may include the synthetic polymers such as carbomers [cross linked polymers of acrylic acid], cellulosic polymers such as hydroxypropyl methylcellulose, methylcellulose, sodium carboxy methylcellulose, and hydroxypropyl cellulose. The different grades of the carbomers available for the topical gel formulation, those are carbopol 910, carbopol 934P. Carbopol 940, carbopol 941, carbopol 1342. These are used in concentration of 0.5% w/w to 2.0% w/w for the purpose. When cellulosic polymers used in the formulation high concentrations is preferred 3% w/w to 15% w/w for the same purpose.

The brief method of manufacturing the said formulation includes the following steps.
1. Dispersing the viscosity increasing agents in the solution of some portion of adjuvant solvent and purified water in a container. Mix these vigorously. This mixing should ensure complete dispersion of the polymer in the solvent. The heating may also allow for the same purpose. But it should be cooled after complete dispersion.
2. Adding slowly the silver suspension to the above jelly mass with mixing. Continue mixing till the silver suspension is uniformly distributed through out the mixture.
3. The other excipients dissolved in the purified water and/or isopropyl alcohol are added to the mixing tank with stirring.
4. Then the pH and the volume are adjusted with the solution of pH adjusting agents and purified water respectively.

It will be appreciated that the present invention, as described above, is not limited to the specific compositions shown nor is it limited to the uses of the compositions described. Modifications in the final compositions and what process is used for making the composition, are all well within the scope of the appended claims. As will be apparent to those skilled in the art, in the light of the foregoing disclosure, many substitutions, alterations, and modifications, as well as different uses of the compositions, are possible in the practice of this invention without departing from the spirit or scope thereof.

### EXAMPLES

### Example-1

Each gram of the gel contains

| | |
|---|---|
| Silver | 0.01 mg (As nano particle) |
| Perfumed Gel base | q.s. |
| Batch size: | 100 kg |

35 kg Propylene Glycol and 18 kg of distilled water were transferred to a steam jacketed planetary mixer. 1.00 kg Carbomor polymer was slowly added under fast stirring. Carbomor polymer was dissolved completely with heating. The gel mass was cooled to room temperature. 10.0 kg of silver suspension of 100-ppm concentration of silver was added to the mixer slowly with mixing till a uniform mass of gel formed. 100 ml of perfume was added to this viscous solution. Remaining 10 kg of the propylene glycol and 4 kg of the isopropyl alcohol were added to this solution with continuous mixing. The pH of the solution was adjusted with sodium hydroxide solution to 5.84. The final weight of the gel was adjusted with the purified water.

### In Process

1. Appearance: Clear, slight pink colored, clear and transparent gel.
2. pH: 5.84

### Example-2

Each gram of the gel contains

| | |
|---|---|
| Silver | 0.02 mg (As nano particle) |
| Perfumed Gel base | q.s. |
| Batch size: | 100 kg |

30 kg Propylene Glycol and 18 kg of distilled water were transferred to the steam jacketed planetary mixer. 1.00 kg Carbomor polymer was slowly added under fast stirring. Carbomor polymer was dissolved completely with heating. The gel mass was cooled to room temperature. 20.0 kg of silver suspension of 100-ppm concentration of silver was added to the mixer slowly with mixing till a uniform mass of gel formed. 100 ml of the perfume was added to this viscous solution. Remaining 10 kg of the propylene glycol and 2 kg of the Polysorbate 80 were added to this solution with continuous mixing. The pH of the solution was adjusted with sodium hydroxide solution to 6.08. The final weight of the gel was adjusted with the purified water.

### In Process

1. Appearance: Clear, slight pink colored, clear and transparent gel.
2. pH: 6.08

### Example-3

Each gram of the gel contains

| | |
|---|---|
| Silver | 0.05 mg (As nano particle) |
| Perfumed Gel base | q.s. |
| Batch size: | 100 kg |

20 kg Propylene Glycol and 10 kg of distilled water were transferred to the steam jacketed planetary mixer. 1.20 kg Carbomor polymer was slowly added under fast stirring. Carbomor polymer was dissolved completely with heating. The gel mass was cooled to room temperature. Added 50.0 kg of the silver suspension of 100-ppm concentration of silver was added to the mixer slowly with mixing till a uniform mass of gel formed. 100 ml of the perfume was added to this viscous solution. Remaining 5 kg of the propylene glycol and 2 kg of the Polysorbate 80 were added to this solution with continuous mixing. The pH of the solution was adjusted with sodium hydroxide solution to 5.90. The final weight of the gel was adjusted with the purified water.

### In Process

1. Appearance: Clear, slight pink colored, clear and transparent gel.
2. pH: 5.90

### Example-4

Each gram of the gel contains

| | |
|---|---|
| Silver | 0.03 mg (As nano particle) |
| Perfumed Gel base | q.s. |
| Batch size: | 100 kg |

20 kg Propylene Glycol and 10 kg of distilled water were transferred to the steam jacketed planetary mixer. 1.20 kg Carbomor polymer was slowly added under fast stirring. Carbomor polymer was dissolved completely with heating. The gel mass was cooled to room temperature. 30.0 kg of silver suspension of 100-ppm concentration of silver was added to the mixer slowly with mixing till a uniform mass of gel formed. 100 ml of the perfume was added to this viscous solution. Remaining 5 kg of the propylene glycol and 4 kg of the Polysorbate 20 were added to this solution with continuous mixing. The pH of the solution was adjusted with sodium hydroxide solution to 5.82. The final weight of the gel was adjusted with the purified water.

### In Process

1. Appearance: Clear, slight pink colored, clear and transparent gel.
2. pH: 5.82

### Example-5

Each gram of the gel contains

| | |
|---|---|
| Silver | 0.02 mg (As nano particle) |
| Perfumed Gel base | q.s. |
| Batch size: | 100 kg |

20 kg Propylene Glycol and 10 kg of distilled water were transferred to the steam jacketed planetary mixer. 1.20 kg Carbomor polymer was slowly added under fast stirring. Carbomor polymer was dissolved completely with heating. The gel mass was cooled to room temperature. 20.0 kg of silver suspension of 100-ppm concentration of silver was added to the mixer slowly with mixing till a uniform mass of gel formed. 100 ml of the perfume was added to this viscous solution. Remaining 5 kg of the propylene glycol and 3 kg of the Polysorbate 40 were added to this solution with continuous mixing. The pH of the solution was adjusted with sodium hydroxide solution to 6.05. The final weight of the gel was adjusted with the purified water.

### In Process

1. Appearance: Clear, slight pink colored, clear and transparent gel.
2. pH: 6.05

### Example-6

Each gram of the gel contains

| | |
|---|---|
| Silver | 0.01 mg (As nano particle) |
| Perfumed Gel base | q.s. |
| Batch size: | 100 kg |

20 kg Propylene Glycol and 10 kg of distilled water were transferred to the steam jacketed planetary mixer. 1.20 kg Carbomor polymer was slowly added under fast stirring. Carbomor polymer was dissolved completely with heating. The gel mass was cooled to room temperature. 10.0 kg of silver suspension of 100-ppm concentration of silver was added to the mixer slowly with mixing till a uniform mass of gel formed. 100 ml of the perfume was added to this viscous solution. Remaining 5 kg of the propylene glycol and 10 kg of the isopropyl alcohol were added to this solution with continuous mixing. The pH of the solution was adjusted with sodium hydroxide solution to 5.80. The final weight of the gel was adjusted with the purified water.

### In Process

1. Appearance: Clear, slight pink colored, clear and transparent gel.
2. pH: 5.80

### Example-7

Each gram of the gel contains

| | |
|---|---|
| Silver | 0.04 mg (As nano particle) |
| Perfumed Gel base | q.s. |
| Batch size: | 100 kg |

20 kg Propylene Glycol and 10 kg of distilled water were transferred to the steam jacketed planetary mixer. 1.20 kg Carbomor polymer was added slowly under fast stirring. Carbomor polymer was dissolved completely with heating. The gel mass was cooled to room temperature. 40.0 kg of silver suspension of 100-ppm concentration of silver was added to the mixer slowly with mixing till a uniform mass of gel formed. 100 ml of the perfume was added to this viscous solution. Remaining 5 kg of the propylene glycol and 3 kg of the PEG-300 were added to this solution with continuous mixing. The pH of the solution was adjusted with sodium hydroxide solution to 5.88. The final weight of the gel was adjusted with the purified water.

### In Process

1. Appearance: Clear, slight pink colored, clear and transparent gel.
2. pH: 5.88

### Example-8

Each gram of the gel contains

| | |
|---|---|
| Silver | 0.05 mg (As nano particle) |
| Perfumed Gel base | q.s |
| Batch size: | 100 kg |

20 kgs Propylene Glycol and 10 kg of distilled water were transferred to the steam jacketed planetary mixer. 1.20 kg Carbomor polymer was slowly added under fast stirring. Carbomor polymer was dissolved completely with heating. The gel mass was cooled to room temperature. 50.0 kg of silver suspension of 100-ppm concentration of silver was added to the mixer slowly with mixing till a uniform mass of gel formed. 100 ml of the perfume was added to this viscous solution. 5.0 kg of the polyethylene glycol, 1 kg of benzyl alcohol and 0.01 kg of propyl gallete were added to this solution with continuous mixing. The pH of the solution was adjusted with sodium hydroxide solution to 5.62. The final weight of the gel was adjusted with the purified water.

### In Process

1. Appearance: Clear, slight pink colored, clear and transparent gel.
2. pH: 5.62

### Example-9

### Stability Study

The three batches of each formulations of above examples were placed in a stability chamber for stability study. The formulations were found to be stable in room temperature (bellow 25° C.) for at least 18 months with a maximum decrease of the potency up to 8% than the initial concentration. On exposure to a higher temperature (more than 40° C.) the product degraded. It found that the product was sensitive to light to some extent. The reports are tabulated bellow.

Each gm of gel contains:

Silver: 0.02 mg as nano-particles
Perfumed gel base: q.s.

TABLE 1
Samples of example 1

| | | 1 Month | 2 Month | 3 Month |
|---|---|---|---|---|
| | | (40° C./ | (40° C./ | (40° C./ |
| Test | Initial | 70% RH) | 70% RH) | 70% RH) |
| Appearance | Clear, Pink | Clear, Pink | Clear, Pink | Clear, Pink |
| | color, | color, | color, | color, |
| | transparent | transparent | transparent | transparent |
| | gel | gel | gel | gel |
| pH | 5.88 | 5.85 | 5.91 | 5.87 |
| % age Assay | 99.86 | 98.17 | 97.69 | 97.21 |
| of Silver (L.A) | | | | |

TABLE 2
Samples of example 2

| | | 1 Month | 2 Month | 3 Month |
|---|---|---|---|---|
| | | (40° C./ | (40° C./ | (40° C./ |
| Test | Initial | 70% RH) | 70% RH) | 70% RH) |
| Appearance | Clear, Pink | Clear, Pink | Clear, Pink | Clear, Pink |
| | color, | color, | color, | color, |
| | transparent | transparent | transparent | transparent |
| | gel | gel | gel | gel |
| pH | 6.12 | 6.08 | 5.92 | 5.93 |
| % age Assay | 98.16 | 97.54 | 96.28 | 96.15 |
| of Silver (L.A) | | | | |

TABLE 3
Samples of example 3

| | | 1 Month | 2 Month | 3 Month |
|---|---|---|---|---|
| | | (40° C./ | (40° C./ | (40° C./ |
| Test | Initial | 70% RH) | 70% RH) | 70% RH) |
| Appearance | Clear, Pink | Clear, Pink | Clear, Pink | Clear, Pink |
| | color, | color, | color, | color, |
| | transparent | transparent | transparent | transparent |
| | gel | gel | gel | gel |
| pH | 6.24 | 6.28 | 6.12 | 6.24 |
| % age Assay | 100.27 | 98.91 | 98.03 | 97.32 |
| of Silver (L.A) | | | | |

### Example-10

### In-Vitro Study

The gel of example 2 as described above was tested for efficacy for the following indications and microorganisms:

### (1) Skin Abscesses

Staphylococcus aureus
Pseudomonas aeruginosa

### (2) Indicator Bacteria

Staphylococcus epidermides
Bacillus pumilus

### (3) Acne

Propionibacterium acnes

### (4) Intestinal Bacteria

Escherichia Coli
Proteus vulgaris
Enterobacter species

The gel was found to be stable at room temperature and soothing skin friendly, non-stainable.

### Procedure:

A standardized sensitivity test was used to evaluate the antibacterial activity of the formulation three confluent lawn plates of standard culture viz. E. coli, P. Aureginasa and S. aureus were prepared by adding 0.2 ml of log phase culture (6 hold) in 25 ml of Muller Hinton agar so as to give final cell density of 1×105 CFU/ml. After solidification of the medium, five wells 98 mm in diameter) were made using a sterile cork bore. Each well was then filled with nanosilver antibacterial formulation Commercially available Silver sulfadiazine formulation was used as positive control, The plates were pre-incubated at 4° C. for 30 min and then transferred to an incubator set at 37° C. Zone of inhibitions around the wells were measured after 18 hours incubation.

### Results:

Results obtained are shown in the Table. These clearly show that silver gel formulations have more potent activity due to increased silver levels. The results are comparable to silver sulfadiazine. It needs to be monitored that the silver concentration in silver sulfadiazine preparations is at least 200 folds that of nanosilver formulations. The present invention formulations show similar activity in very small concentration. Thus very small concentration of silver as nano particle dispersion form can able to produce its reported action.

TABLE 04

| | Zone of Inhibition (mm) | | | Silver |
|---|---|---|---|---|
| FORMULATION | E. coli | P. Aureginasa | S. aureus | (mg/g) |
| Silver gel | 19 | 15 | 15 | 0.016 |
| (example -2) | | | | |
| Silver gel | 22 | 16 | 21 | 0.018 |
| (example -2) | | | | |
| Silver | 23 | 16 | 21 | 3.5 |
| Sulfadiazine | | | | |
| (marketed | | | | |
| product) | | | | |

1. A topical semisolid (gel) formulation comprising,
a) a therapeutically effective amount of silver nanoparticles of size in the range of 0.1 nanometer to 150 nanometer having a mass of about 0.001% w/w to about 1% w/w of the total mass of the formulation;
b) at least one gel forming agent (viscosity enhancing polymer) having a mass of about 0.5% w/w to about 25% w/w of the total mass of the formulation;
c) at least one co-solvent (penetrating enhancing agents) having a mass of about 25% w/w to 60% w/w of the total mass of the formulation;
d) purified water having a mass of about 1% w/w to 50% of the total mass of the formulation;
e) optionally at least one preservative having a mass of about 0.001% w/w to 2.0% w/w of the total mass of the formulation;
f) optionally at least one antioxidant having a mass of about 0.001% w/w to 2.0% w/w of the total mass of the formulation;
g) optionally at least one pH adjusting agent having a mass of about 0.01% w/w to 1.0% w/w of the total mass of the formulation;
h) optionally at least one fragrance having a mass of about 0.01% w/w to 1.0% w/w of the total mass of the formulation.

2. A topical semisolid (gel) formulation of claim 1, wherein silver is in the form of a suspension.

3. A topical semisolid (gel) formulation of claim 1, wherein the semisolid formulation is used for local administration to the infected area of the skin or the oral cavity on the microbial infections in cuts, wound, burns and other topical microbial infection.

4. A topical semisolid (gel) formulation of claim 1, wherein the gel forming agent is a compound selected from the group consisting of carbomers [cross linked polymers of acrylic acid] and cellulosic polymers.

5. A topical semisolid (gel) formulation of claim 1, wherein the cosolvent is a compound selected from the group consisting of Propylene glycol, polyethylene glycols, cremaphors, ethyl alcohol, isopropyl alcohol and Polysorbate.

6. A topical semisolid (gel) formulation of claim 1, wherein the preservative is a compound selected from the group consisting of chlorocresol, methyl paraben, propyl paraben, thiomarsal, sorbic acid, potassium sorbate and benzyl alcohol.

7. A topical semisolid (gel) formulation of claim 1, wherein the antioxidant is a compound selected from the group consisting of Butylated hydroxy anisole (BHA), Butylated hydroxy toluene (BHT), sodium metabisulfite, sodium sulfite, sodium bisulfite and propyl gallate.

8. A topical semisolid (gel) formulation of claim 1, wherein the pH adjusting agent is a compound selected from the group consisting of diethanolamine, triethanolamine, sodium hydroxide, hydrochloric acid, citric acid and mono basic sodium phosphate.

9. A topical semisolid (gel) formulation of claim 1, wherein the pH of the formulation is in the range of 4.5 to 6.5.

10. A process for preparing a topical semisolid (gel) formulation of claim 1 comprising the following steps
a) Dispersing the gel forming agent in the mixture of cosolvent and purified water in a container and mixing vigorously at the temperature of 25 to 70° C. and after complete dispersion cooling the solution to get the jelly mass,
b) Adding the silver suspension to the jelly mass with mixing till the silver suspension is uniformly distributed through out the content of the mixer to get silver jelly,
c) Dissolving preservative, antioxidant and fragrance in the purified water and/or isopropyl alcohol to get excipients solution,
d) Adding excipients solution to the mixing tank having silver jelly with stirring and adjusting the pH and the volume with the solution of pH adjusting agents and purified water respectively.

11. (canceled)

12. A topical semisolid (gel) formulation of claim 4 wherein the cellolusic polymer is selected from the group consisting of hydroxypropyl methylcellulose, methylcellulose, sodium carboxy methylcellulose, and hydroxypropyl cellulose.
